(19) United States
(12) Patent Application Publication
(10) Pub. No.: US 2020/0170975 A1
(43) Pub. Date: Jun. 4, 2020

(54) METHODS OF TREATING DISEASE WITH DICHLORPHENAMIDE

(71) Applicant: Strongbridge Dublin Limited, Trevose, PA (US)

(72) Inventor: Fredric Jay COHEN, Washington Crossing, PA (US)

(21) Appl. No.: 16/540,456

(22) Filed: Aug. 14, 2019

Related U.S. Application Data
(62) Division of application No. 16/205,602, filed on Nov. 30, 2018.

(51) Int. Cl. A61K 31/18

(52) U.S. Cl. CPC A61K 31/18

(57) ABSTRACT

Provided herein is a method for administering dichlorphenamide, or a pharmaceutically acceptable salt thereof, to a subject in need thereof, wherein the subject is also being administered a therapeutically effective amount of a drug chosen from a P-gp substrate, BCRP substrate, OAT2 substrate, OAT4 substrate, OCT1 substrate, MATE1 substrate, MATE2-K substrate, and combinations thereof. The method comprises administering to the subject a therapeutically effective amount of dichlorphenamide, or a pharmaceutically acceptable salt thereof. The therapeutically effective amount of the drug is not adjusted relative to a subject who is not being administered dichlorphenamide.

The present disclosure relates to new compositions, and their application as pharmaceuticals for treating disease. Methods of treating hyperkalemic periodic paralysis, hypokalemic periodic paralysis and other diseases in a human or animal subject are also provided.

Numerous endo- and xenobiotics including many drugs are organic anions or cations. Their disposition and elimination depend on the proper function of multispecific drug transporters that belong to two major superfamilies: solute carrier (SLC) transporters and ATP-binding cassette (ABC) transporters. Although most are capable of bidirectional transport, in general, ABC transporters are responsible for efflux of substrates, while SLC transporters mediate uptake of substrates into cells.

Dichlorphenamide (Keveyis®, Daranide™) is a carbonic anhydrase inhibitor approved for treating primary hyperkalemic periodic paralysis, primary hypokalemic periodic paralysis, and related variants, and has been used to treat intraocular pressure (TOP). Dichlorphenamide was introduced by Merck in 1950's to treat glaucoma. Dichlorphenamide is now available as immediate-release tablets for oral administration, each containing 50 mg dichlorphenamide.

The present disclosure provides method for administering dichlorphenamide, or a pharmaceutically acceptable salt thereof, to a subject in need thereof, wherein the subject is also being administered a therapeutically effective amount of a drug chosen from a P-gp substrate, BCRP substrate, OAT2 substrate, OAT4 substrate, OCT1 substrate, MATE1 substrate, MATE2-K substrate, and combinations thereof. The method comprises administering to the subject a therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

The present disclosure further provides a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from a P-gp substrate, BCRP substrate, OAT2 substrate, OAT4 substrate, OCT1 substrate, MATE1 substrate, MATE2-K substrate, and combinations thereof, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

These and other aspects of the invention will be apparent upon reference to the following detailed description. To this end, various references are set forth herein which describe in more detail certain background information, procedures, compounds, and/or compositions, and are each hereby incorporated by reference in their entirety.

### BRIEF SUMMARY OF THE DRAWINGS

FIG. 1 shows the expression of OATPs in selected human epithelial cells. OATP1A2 expression in cholangiocytes has been demonstrated but not yet localized to a distinct cell membrane. “A” refers to apical and “B” to basal/basolateral. This figure is adapted from FIG. 1 of Roth et al., “OATPs, OATs and OCTs: the organic anion and cation transporters of the SLCO and SLC22A gene superfamilies,” British Journal of Pharmacology (2012) 165:1260-1297, incorporated herein by reference in its entirety.

FIG. 2 shows expression of OATs in different human epithelia. OAT1 localization in the choroid plexus and OAT2 localization in the liver is inferred from rodent data. “A” refers to apical and “B” to basal/basolateral. This figure is adapted from FIG. 3 of Roth et al. 2012.

FIG. 3 shows expression of OCTs in human epithelial cells. Localization of OCTN1 in the kidney is concluded based on rodent data. “A” refers to apical and “B” to basal/basolateral. This figure is adapted from FIG. 5 of Roth et al. 2012.

### DETAILED DESCRIPTION

Dichlorphenamide is a dichlorinated benzenedisulfonamide, known chemically as 4,5-dichloro-1,3-benzenedisulfonamide. Its empirical formula is C₆H₆C₁₂N₂O₄S₂ and its structural formula is:

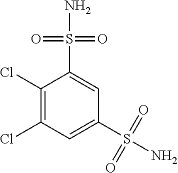 [SMILES: NS(=O)(=O)c1cc(Cl)c(Cl)c(S(N)(=O)=O)c1]

Dichlorphenamide USP is a white or practically white, crystalline compound with a molecular weight of 305.16 g/mol. It is very slightly soluble in water but soluble in dilute solutions of sodium carbonate and sodium hydroxide. Dilute alkaline solutions of dichlorphenamide are stable at room temperature. Dichlorphenamide is storage-stable for at least 36 months.

A formulation of dichlorphenamide has been previously reported in the United States Food and Drug Administration (FDA) approved drug label for Keveyis®, which is indicated for treating primary hyperkalemic periodic paralysis (“hyper”), primary hypokalemic periodic paralysis (“hypo”), and related variants, a heterogenous group of conditions for which responses may vary. The initial dose is 50 mg/day twice daily (bis in diem, BID), which may be adjusted at weekly intervals up to 200 mg/day.

Dichlorphenamide is a carbonic anhydrase inhibitor. The precise mechanism by which dichlorphenamide exerts its therapeutic effects in patients with periodic paralysis is unknown. It is hypothesized that dichlorphenamide modulates pH, which affects the resting membrane potential on muscle surfaces. For both hypo and hyper, the muscles have lost their charge and stop responding.

When introducing elements of the present disclosure or the preferred embodiment(s) thereof, the articles “a”, “an”, “the” and “said” are intended to mean that there are one or more of the elements. The terms “comprising”, “including” and “having” are inclusive and mean that there may be additional elements other than the listed elements.

The term “and/or” when in a list of two or more items, means that any of the listed items can be employed by itself or in combination with one or more of the listed items. For example, the expression “A and/or B” means either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where ni and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about” qualifies the numerical values that it modifies, denoting such a value as variable within a margin of error. When no margin of error, such as a standard deviation to a mean value given in a chart or table of data, is recited, the term “about” means that range which would encompass the recited value and the range which would be included by rounding up or down to that figure, considering significant figures.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate headache, whereas a second patient may find headache tolerable but is not able to tolerate vomiting, whereas for a third patient, either headache alone or vomiting alone is tolerable, but the patient is not able to tolerate the combination of headache and vomiting, even if the severity of each is less than when experienced alone.

As used herein, an “adverse event” is an untoward medical occurrence associated with treatment with a substrate of a drug transporter.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine.

Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While the disclosed compounds may be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds may be a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

In addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

In certain embodiments, the subject may receive a dose of between 50 mg twice daily and to 100 mg twice daily. In certain embodiments, the dose is 50 mg once daily. In certain embodiments, the dose is 50 mg once every other day. In certain embodiments, the dose is 25 mg once daily. In certain embodiments, the dose is 25 mg once every other day.

In certain embodiments, the therapeutically effective amount of the dichlorphenamide, or a pharmaceutically acceptable salt thereof, is between 25 mg and 200 mg per day.

In certain embodiments, the therapeutically effective amount of the dichlorphenamide, or a pharmaceutically acceptable salt thereof, is 50 mg twice daily.

In certain embodiments, the dichlorphenamide, or a pharmaceutically acceptable salt thereof, is administered via a titration scheme that comprises the up-titration of the dichlorphenamide, or a pharmaceutically acceptable salt thereof, at weekly intervals until a modified dose is administered. In certain embodiments, the modified dose of the dichlorphenamide, or a pharmaceutically acceptable salt thereof, is 200 mg.

The amount of active ingredient that may be combined with the carrier materials to produce a single dosage form will vary depending upon the host treated and the mode of administration.

The compounds can be administered in various modes, e.g. orally, topically, or by injection. The precise amount of compound administered to a patient will be the responsibility of the attendant physician. The specific dose level for any patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few min to four weeks.

In certain embodiments, the disease chosen from primary hyperkalemic periodic paralysis, primary hypokalemic periodic paralysis, and related variants; Aland Island eye disease atrial fibrillation, Brugada syndrome, cardiomyopathy, cerebellar syndrome, cone-rod dystrophy, cystoid macular edema of retinitis pigmentosa, Dravet syndrome, epilepsy, epileptic encephalopathy, episodic ataxia, myokymia syndrome, episodic pain syndrome, hemiplegic migraine, febrile seizures, heart block, intracranial hypertension, long QT syndrome, neuropathy, night blindness, paroxysmal exercise-induced dyskinesia, Rett syndrome, sick sinus syndrome, spinocerebellar ataxia, sudden infant death syndrome (SIDS), Timothy syndrome, and ventricular fibrillation.

In certain embodiments, the disease chosen from primary hyperkalemic periodic paralysis, primary hypokalemic periodic paralysis, and related variants. In certain embodiments, the disease primary hyperkalemic periodic paralysis. In certain embodiments, the disease is primary hypokalemic periodic paralysis. In certain embodiments, the disease is a related variant to primary hyperkalemic periodic paralysis. In certain embodiments, the disease is a related variant to primary hypokalemic periodic paralysis.

In certain embodiments, the disease is Aland Island eye disease.

In certain embodiments, the disease is atrial fibrillation, such as familial atrial fibrillation.

In certain embodiments, the disease is Brugada syndrome, such as type 1 or type 3.

In certain embodiments, the disease is cardiomyopathy, such as dilated cardiomyopathy.

In certain embodiments, the disease is cerebellar syndrome in phosphomannomutase 2 (PMM2) deficiency, a congenital disorder of glycosylation.

In certain embodiments, the disease is cone-rod dystrophy, such as X-linked cone-rod dystrophy.

In certain embodiments, the disease is cystoid macular edema of retinitis pigmentosa.

In certain embodiments, the disease is Dravet syndrome.

In certain embodiments, the disease is epilepsy, such as generalized epilepsy, epilepsy type two, or epilepsy with febrile seizures.

In certain embodiments, the disease is epileptic encephalopathy, early infantile epileptic encephalopathy, which is an autosomal dominant form of the disease.

In certain embodiments, the disease is episodic ataxia, such as type 1, type 2, or type 5, or myokymia syndrome

In certain embodiments, the disease is episodic pain syndrome, such as familial episodic pain syndrome.

In certain embodiments, the disease is hemiplegic migraine types, familial hemiplegic migraine types 1 and 3.

In certain embodiments, the disease is febrile seizures, such as familial febrile seizures.

In certain embodiments, the disease is heart block, such as nonprogressive heart block, and progressive heart block type IA.

In certain embodiments, the disease is intracranial hypertension, such as idiopathic intracranial hypertension.

In certain embodiments, the disease is long QT syndrome-3

In certain embodiments, the disease is neuropathy, hereditary neuropathy, sensory neuropathy, and autonomic neuropathy type VII.

In certain embodiments, the disease is night blindness, such as congenital stationary night blindness, and X-linked night blindness.

In certain embodiments, the disease is paroxysmal exercise-induced dyskinesia.

In certain embodiments, the disease is Rett syndrome.

In certain embodiments, the disease is sick sinus syndrome.

In certain embodiments, the disease is spinocerebellar ataxia, such as spinocerebellar ataxia type 6.

In certain embodiments, the disease is sudden infant death syndrome (SIDS).

In certain embodiments, the disease is Timothy syndrome.

In certain embodiments, the disease is ventricular fibrillation, such as familial ventricular fibrillation.

In certain embodiments, dichlorphenamide is not an inhibitor of P-gp, BCRP, OAT2, OAT4, OCT1, MATE1 or MATE2-K. In certain embodiments, dichlorphenamide is not an inhibitor of P-gp. In certain embodiments, dichlorphenamide is not an inhibitor of BCRP. In certain embodiments, dichlorphenamide is not an inhibitor of OAT2. In certain embodiments, dichlorphenamide is not an inhibitor of OAT4. In certain embodiments, dichlorphenamide is not an inhibitor of OCT1. In certain embodiments, dichlorphenamide is not an inhibitor of MATE1. In certain embodiments, dichlorphenamide is not an inhibitor of MATE2-K.

The human organic anion and cation transporters are classified within two Solute Carrier (SLC) superfamilies. The Solute Carrier Organic Anion (SLCO, formerly SLC21A) superfamily consists of organic anion transporting polypeptides (OATPs), while the organic anion transporters (OATs) and the organic cation transporters (OCTs) are classified in the solute carrier family 22A (SLC22A) superfamily. Individual members of each superfamily are expressed in epithelia throughout the body, where they absorb, distribute and eliminate drugs. Substrates of OATPs are large hydrophobic organic anions, while OATs transport smaller and more hydrophilic organic anions and OCTs transport organic cations. In addition to endogenous substrates, such as steroids, hormones and neurotransmitters, these proteins transport numerous drugs and other xenobiotics are transported, including statins, antivirals, antibiotics and anticancer drugs.

Expression of OATPs, OATs and OCTs can be regulated at the protein or transcriptional level and varies within each family by protein and tissue type. All three superfamilies consist of 12 transmembrane domain proteins with intracellular termini. Although no crystal structures have yet been determined, homology modelling and mutation experiments have explored the mechanism of substrate recognition and transport. Several polymorphisms identified in superfamily members have been shown to affect pharmacokinetics of their drug substrates, confirming the importance of these drug transporters for efficient pharmacological therapy.

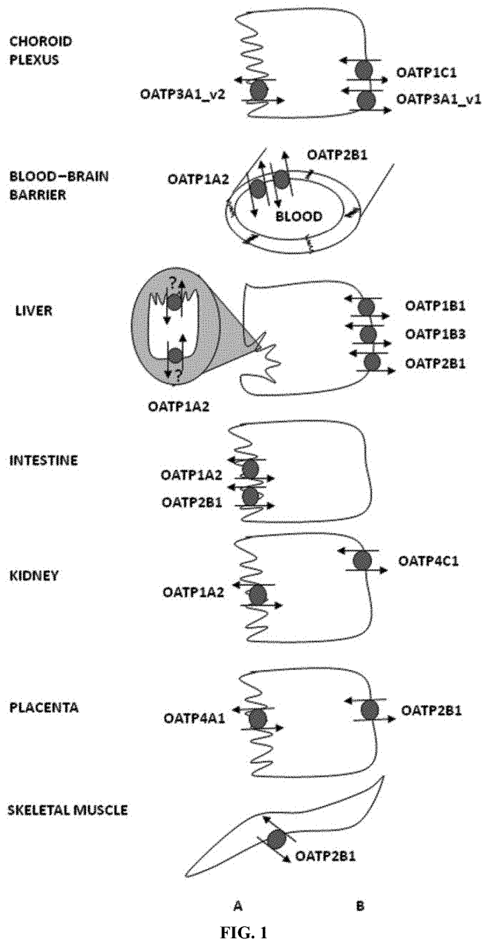

An organic-anion-transporting polypeptide (OATP) is a membrane transport protein or “transporter” that mediates the transport of mainly organic anions across the cell membrane (FIG. 1). Therefore, OATPs are the gatekeepers in the lipid bilayer of the cell membrane. OATP1B1, OATP1B3 and OCT1 are expressed on the sinusoidal membrane of hepatocytes and aid the accumulation of endogenous and xenobiotic compounds into hepatocytes for further metabolism or excretion into the bile. As well as expression in the liver, OATPs are expressed in many other tissues on basolateral and apical membranes, transporting anions, neutral and cationic compounds. They transport an extremely diverse range of drug compounds, including anti-cancer, antibiotic, lipid lowering drugs, anti-diabetic drugs, toxins and poisons.

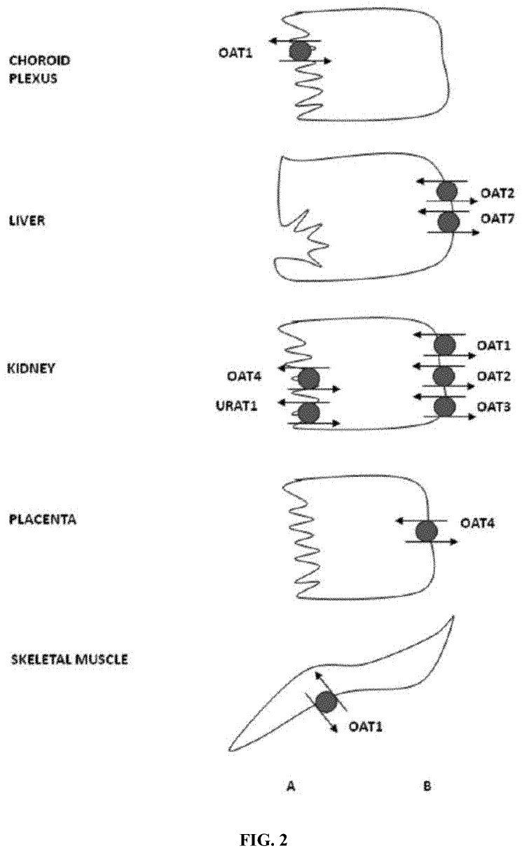

Organic anion transporters (OATs in humans, Oats in rodents) are another family of multispecific transporters and are encoded by the SLC22/S1c22 gene superfamily. They mediate the transport of a diverse range of low molecular weight substrates including steroid hormone conjugates, biogenic amines, various drugs and toxins. See FIG. 2.

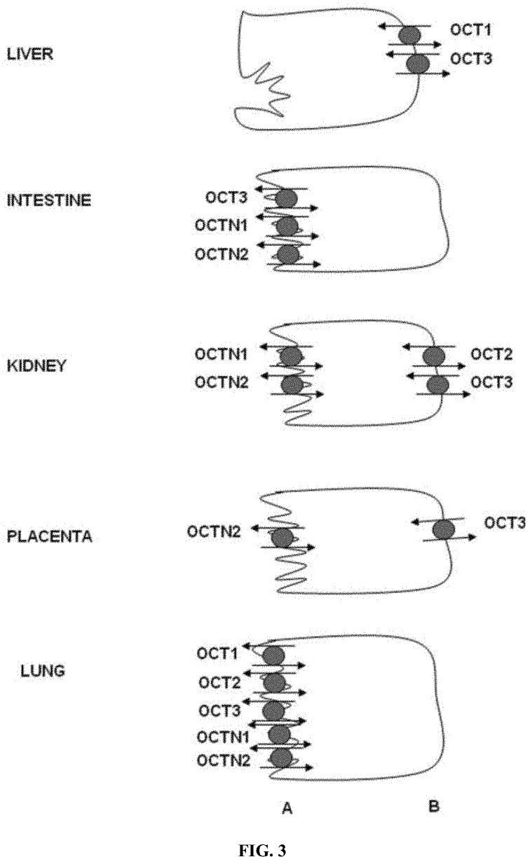

In addition to the OATs described above, the SLC22A family also contains the organic cation transporters (OCT1, OCT2 and OCT3) and the organic cation and carnitine transporters (OCT6, OCTN1 and OCTN2). Like the OATPs and OATs, OCTs are multispecific uptake transporters expressed in numerous epithelia throughout the body. See FIG. 3.

OAT2 (Solute carrier family 22 member 7) is involved in the sodium-independent transport and excretion of organic anions. This integral membrane protein is localized to the basolateral membrane of the kidney.

In certain embodiments, there is provided a method for administering dichlorphenamide, or a pharmaceutically acceptable salt thereof, to a subject in need thereof, wherein the subject is also being administered a therapeutically effective amount of an OAT2 substrate. The method comprises administering to the subject a therapeutically effective amount of dichlorphenamide, or a pharmaceutically acceptable salt thereof, wherein the therapeutically effective amount of the OAT2 substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from an OAT2 substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the OAT2 substrate is chosen from dinoprostone, cimetidine, aminohippuric acid, cyclic adenosine monophosphate (cAMP), valproic acid, salicylic acid, glutaric acid, allopurinol, zalcitabine, acetylsalicylic acid, indomethacin, fluorouracil, docetaxel, tegafur-uracil, and combinations thereof.

In certain embodiments, the OAT2 substrate is dinoprostone. In certain embodiments, the OAT2 substrate is cimetidine. In certain embodiments, the OAT2 substrate is aminohippuric acid. In certain embodiments, the OAT2 substrate is cyclic adenosine monophosphate (cAMP). In certain embodiments, the OAT2 substrate is valproic acid. In certain embodiments, the OAT2 substrate is salicylic acid. In certain embodiments, the OAT2 substrate is glutaric acid. In certain embodiments, the OAT2 substrate is allopurinol. In certain embodiments, the OAT2 substrate is zalcitabine. In certain embodiments, the OAT2 substrate is acetylsalicylic acid. In certain embodiments, the OAT2 substrate is indomethacin. In certain embodiments, the OAT2 substrate is fluorouracil. In certain embodiments, the OAT2 substrate is docetaxel. In certain embodiments, the OAT2 substrate is tegafur-uracil.

OAT4 (Solute carrier family 22 member 11) is involved in the sodium-independent transport and excretion of organic anions. This integral membrane protein and is found mainly in the kidney and in the placenta, where it may act to prevent potentially harmful organic anions from reaching the fetus.

In certain embodiments, there is provided a method for administering dichlorphenamide, or a pharmaceutically acceptable salt thereof, to a subject in need thereof, wherein the subject is also being administered a therapeutically effective amount of an OAT4 substrate. The method comprises administering to the subject a therapeutically effective amount of dichlorphenamide, or a pharmaceutically acceptable salt thereof, wherein the therapeutically effective amount of the OAT4 substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from an OAT4 substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the OAT4 substrate is chosen from aminohippuric acid, conjugated estrogens, and combinations thereof. In certain embodiments, the OAT4 substrate is aminohippuric acid. In certain embodiments, the OAT4 substrate is conjugated estrogens.

OCT1 (Solute carrier family 22 member 1, SLC22A1), is a protein that in humans is encoded by the gene SLC22A1. Polyspecific organic cation transporters in the liver, kidney, intestine, and other organs are critical for elimination of many endogenous small organic cations as well as a wide array of drugs and environmental toxins. This gene is one of three similar cation transporter genes located in a cluster on chromosome 6. The encoded protein contains twelve putative transmembrane domains and is a plasma integral membrane protein. Two transcript variants encoding two different isoforms have been found for this gene, but only the longer variant encodes a functional transporter.

In certain embodiments, there is provided a method for administering dichlorphenamide, or a pharmaceutically acceptable salt thereof, to a subject in need thereof, wherein the subject is also being administered a therapeutically effective amount of an OCT1 substrate. The method comprises administering to the subject a therapeutically effective amount of dichlorphenamide, or a pharmaceutically acceptable salt thereof, wherein the therapeutically effective amount of the OCT1 substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from an OCT1 substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the OCT1 substrate is chosen from ganciclovir, acyclovir, choline, amantadine, verapamil, quinine, cimetidine, dexchlorpheniramine, choline salicylate, rocuronium, phenformin, metformin, thiamine, dopamine, dancuronium, epinephrine, imatinib, norepinephrine, acetylcholine, spermine, spermidine, tubocurarine, buformin, cytarabine, pramipexole, agmatine, lamivudine, nafamostat, and combinations thereof.

In certain embodiments, the OCT1 substrate is ganciclovir. In certain embodiments, the OCT1 substrate is acyclovir. In certain embodiments, the OCT1 substrate is choline. In certain embodiments, the OCT1 substrate is amantadine. In certain embodiments, the OCT1 substrate is verapamil. In certain embodiments, the OCT1 substrate is quinine. In certain embodiments, the OCT1 substrate is cimetidine. In certain embodiments, the OCT1 substrate is dexchlorpheniramine. In certain embodiments, the OCT1 substrate is choline salicylate. In certain embodiments, the OCT1 substrate is rocuronium. In certain embodiments, the OCT1 substrate is phenformin. In certain embodiments, the OCT1 substrate is metformin. In certain embodiments, the OCT1 substrate is thiamine. In certain embodiments, the OCT1 substrate is dopamine. In certain embodiments, the OCT1 substrate is dancuronium. In certain embodiments, the OCT1 substrate is epinephrine. In certain embodiments, the OCT1 substrate is imatinib. In certain embodiments, the OCT1 substrate is norepinephrine. In certain embodiments, the OCT1 substrate is acetylcholine. In certain embodiments, the OCT1 substrate is spermine. In certain embodiments, the OCT1 substrate is spermidine. In certain embodiments, the OCT1 substrate is tubocurarine. In certain embodiments, the OCT1 substrate is buformin. In certain embodiments, the OCT1 substrate is cytarabine. In certain embodiments, the OCT1 substrate is pramipexole. In certain embodiments, the OCT1 substrate is agmatine. In certain embodiments, the OCT1 substrate is lamivudine. In certain embodiments, the OCT1 substrate is nafamostat.

Other transporters include P-gp, BCRP, MATE1, and MATE2-K, which are expressed on the apical membrane of several tissues. P-gp and BCRP are expressed in the luminal membrane of enterocytes, endothelial cells in the brain, the brush border membrane of renal proximal tubules and the canalicular membrane of hepatocytes where they limit the intestinal absorption, blood-brain barrier penetration and aid excretion into the bile and urine.

Permeability glycoprotein 1 (P-glycoprotein 1, P-gp, Pgp, multidrug resistance protein 1 (MDR1), ATP-binding cassette sub-family B member 1 (ABCB1), or cluster of differentiation 243 (CD243)) pumps many foreign substances out of cells. P-gp is extensively distributed and expressed in the intestinal epithelium where it pumps xenobiotics (such as toxins or drugs) back into the intestinal lumen, in liver cells where it pumps them into bile ducts, in the cells of the proximal tubule of the kidney where it pumps them into urinary filtrate (in the proximal tubule), and in the capillary endothelial cells composing the blood-brain barrier and blood-testis barrier, where it pumps them back into the capillaries. Some cancer cells also express large amounts of P-gp, further amplifying that effect and rendering these cancers multidrug resistant. Many drugs and some foods incidentally inhibit P-gp.

In certain embodiments, there is provided a method for administering dichlorphenamide, or a pharmaceutically acceptable salt thereof, to a subject in need thereof, wherein the subject is also being administered a therapeutically effective amount of a P-gp substrate. The method comprises administering to the subject a therapeutically effective amount of dichlorphenamide, or a pharmaceutically acceptable salt thereof, wherein the therapeutically effective amount of the P-gp substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from a P-gp substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the P-gp substrate is chosen from bilastine, brigatinib, dasabuvir, delafloxacine, naldemedine, vinflunine, amrubicin, brentuximab, fostamatinib, celecoxib, and combinations thereof.

In certain embodiments, the P-gp substrate is bilastine. In certain embodiments, the P-gp substrate is brigatinib. In certain embodiments, the P-gp substrate is dasabuvir. In certain embodiments, the P-gp substrate is delafloxacine. In certain embodiments, the P-gp substrate is naldemedine. In certain embodiments, the P-gp substrate is vinflunine. In certain embodiments, the P-gp substrate is amrubicin. In certain embodiments, the P-gp substrate is brentuximab. In certain embodiments, the P-gp substrate is fostamatinib. In certain embodiments, the P-gp substrate is celecoxib.

Breast cancer resistance protein (BCRP, ATP-binding cassette sub-family G member 2 (ABCG2), or cluster of differentiation w338 (CDw338)) is a xenobiotic transporter which contributes to multidrug resistance to chemotherapeutic agents, including mitoxantrone and camptothecin analogues. Early observations of significant ABCG2-mediated resistance to anthracyclines were subsequently attributed mutations encountered in vitro but not in nature or the clinic. BCRP is significantly expressed in the placenta, and in the fetus from xenobiotics in the maternal circulation. BCRP has also blocks absorption at the apical membrane of the intestine, at the blood-testis barrier, the blood-brain barrier, and the membranes of hematopoietic progenitor and other stem cells. At the apical membranes of the liver and kidney, it enhances excretion of xenobiotics. In the lactating mammary gland, it excretes vitamins such as riboflavin and biotin into milk. In the kidney and gastrointestinal tract, it excretes urate.

In certain embodiments, there is provided a method for administering dichlorphenamide, or a pharmaceutically acceptable salt thereof, to a subject in need thereof, wherein the subject is also being administered a therapeutically effective amount of a BCRP substrate. The method comprises administering to the subject a therapeutically effective amount of dichlorphenamide, or a pharmaceutically acceptable salt thereof, wherein the therapeutically effective amount of the BCRP substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from a BCRP substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the BCRP substrate is chosen from cobimetinib, ledipasvir, gefitinib, pravastatin, imatinib, sorafenib, sulfasalazine, dasatinib, nilotinib, teriflunomide, vemurafenib, ponatinib, dabrafenib, afatinib, velpatasvir, simeprevir, voxilaprevir, enasidenib, pibrentasvir, glecaprevir, bemaciclib, brigatinib, rucaparib, baricitinib, topotecan, glyburide, doxarubin, mitoxantrone, prazosin, lamivudine, irinotecan, etoposide, actinomycin, conjugated estrogens, cerivastatin, testosterone, tamoxifen, sumatriptan, daunorubicin, folic acid, alvocidib, vincristine, teniposide, nitrofurantoin, ivermectin, camptothecin, riluzole, cladribine, clofarabine, oxaliplatin, pitavastatin, pazopanib, leflunomide, apixaban, ezetimibe, fluorouracil, mycophenolate mofetil, cisplatin, carboplatin, rosuvastatin, paclitaxel, docetaxel, sofosbuvir, lenvatnib, idelalisib, osimertinib, riociguat, venetoclax, ombitasvir, delafloxacin, copanlisib, dolutegravir, ertugliflozin, moxidectin, lusutrombopag, talazoparib, and combinations thereof.

In certain embodiments, the BCRP substrate is cobimetinib. In certain embodiments, the BCRP substrate is ledipasvir. In certain embodiments, the BCRP substrate is gefitinib. In certain embodiments, the BCRP substrate is pravastatin. In certain embodiments, the BCRP substrate is imatinib. In certain embodiments, the BCRP substrate is sorafenib. In certain embodiments, the BCRP substrate is sulfasalazine. In certain embodiments, the BCRP substrate is dasatinib. In certain embodiments, the BCRP substrate is nilotinib. In certain embodiments, the BCRP substrate is teriflunomide. In certain embodiments, the BCRP substrate is vemurafenib. In certain embodiments, the BCRP substrate is ponatinib. In certain embodiments, the BCRP substrate is dabrafenib. In certain embodiments, the BCRP substrate is afatinib. In certain embodiments, the BCRP substrate is velpatasvir. In certain embodiments, the BCRP substrate is simeprevir. In certain embodiments, the BCRP substrate is voxilaprevir. In certain embodiments, the BCRP substrate is enasidenib. In certain embodiments, the BCRP substrate is pibrentasvir. In certain embodiments, the BCRP substrate is glecaprevir. In certain embodiments, the BCRP substrate is bemaciclib. In certain embodiments, the BCRP substrate is brigatinib. In certain embodiments, the BCRP substrate is rucaparib. In certain embodiments, the BCRP substrate is baricitinib. In certain embodiments, the BCRP substrate is topotecan. In certain embodiments, the BCRP substrate is glyburide. In certain embodiments, the BCRP substrate is doxarubin. In certain embodiments, the BCRP substrate is mitoxantrone. In certain embodiments, the BCRP substrate is prazosin. In certain embodiments, the BCRP substrate is lamivudine. In certain embodiments, the BCRP substrate is irinotecan. In certain embodiments, the BCRP substrate is etoposide. In certain embodiments, the BCRP substrate is actinomycin. In certain embodiments, the BCRP substrate is conjugated estrogens. In certain embodiments, the BCRP substrate is cerivastatin. In certain embodiments, the BCRP substrate is testosterone. In certain embodiments, the BCRP substrate is tamoxifen. In certain embodiments, the BCRP substrate is sumatriptan. In certain embodiments, the BCRP substrate is daunorubicin. In certain embodiments, the BCRP substrate is folic acid. In certain embodiments, the BCRP substrate is alvocidib. In certain embodiments, the BCRP substrate is vincristine. In certain embodiments, the BCRP substrate is teniposide. In certain embodiments, the BCRP substrate is nitrofurantoin. In certain embodiments, the BCRP substrate is ivermectin. In certain embodiments, the BCRP substrate is camptothecin. In certain embodiments, the BCRP substrate is riluzole. In certain embodiments, the BCRP substrate is cladribine. In certain embodiments, the BCRP substrate is clofarabine. In certain embodiments, the BCRP substrate is oxaliplatin. In certain embodiments, the BCRP substrate is pitavastatin. In certain embodiments, the BCRP substrate is pazopanib. In certain embodiments, the BCRP substrate is leflunomide. In certain embodiments, the BCRP substrate is apixaban. In certain embodiments, the BCRP substrate is ezetimibe. In certain embodiments, the BCRP substrate is fluorouracil. In certain embodiments, the BCRP substrate is mycophenolate mofetil. In certain embodiments, the BCRP substrate is cisplatin. In certain embodiments, the BCRP substrate is carboplatin. In certain embodiments, the BCRP substrate is rosuvastatin. In certain embodiments, the BCRP substrate is paclitaxel. In certain embodiments, the BCRP substrate is docetaxel. In certain embodiments, the BCRP substrate is sofosbuvir. In certain embodiments, the BCRP substrate is lenvatnib. In certain embodiments, the BCRP substrate is idelalisib. In certain embodiments, the BCRP substrate is osimertinib. In certain embodiments, the BCRP substrate is riociguat. In certain embodiments, the BCRP substrate is venetoclax. In certain embodiments, the BCRP substrate is ombitasvir. In certain embodiments, the BCRP substrate is delafloxacin. In certain embodiments, the BCRP substrate is copanlisib. In certain embodiments, the BCRP substrate is dolutegravir. In certain embodiments, the BCRP substrate is ertugliflozin. In certain embodiments, the BCRP substrate is moxidectin. In certain embodiments, the BCRP substrate is lusutrombopag. In certain embodiments, the BCRP substrate is talazoparib.

Multidrug and toxin extrusion transporter 1 (MATE1, solute carrier family 47, member 1 (SLC47A1)) and MATE2-K are primarily expressed on the luminal (apical) membrane of the proximal tubular cells and excrete cations and zwitterions into urine. MATE2 and its splice variant MATE2-K are proton antiporters are polyspecific efflux transporters of diverse substrates, primarily of organic cations. MATE1 and MATE2-K may function with OCT transporters expressed on the canalicular membranes of hepatocytes and the basolateral membranes of proximal tubules to mediate excretion.

In certain embodiments, there is provided a method for administering dichlorphenamide, or a pharmaceutically acceptable salt thereof, to a subject in need thereof, wherein the subject is also being administered a therapeutically effective amount of a MATE1 substrate. The method comprises administering to the subject a therapeutically effective amount of dichlorphenamide, or a pharmaceutically acceptable salt thereof, wherein the therapeutically effective amount of the MATE1 substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from a MATE1 substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the MATE1 substrate is chosen from cimetidine, abemacicilib, levofloxacin, ciprofloxacin, topotecan, metformin, cephalexin, acyclovir, cefradine, estrone sulfate, ganciclovir, guanidine, procainamide, and combinations thereof.

In certain embodiments, the MATE1 substrate is cimetidine. In certain embodiments, the MATE1 substrate is abemacicilib. In certain embodiments, the MATE1 substrate is levofloxacin. In certain embodiments, the MATE1 substrate is ciprofloxacin. In certain embodiments, the MATE1 substrate is topotecan. In certain embodiments, the MATE1 substrate is metformin. In certain embodiments, the MATE1 substrate is cephalexin. In certain embodiments, the MATE1 substrate is acyclovir. In certain embodiments, the MATE1 substrate is cefradine. In certain embodiments, the MATE1 substrate is estrone sulfate. In certain embodiments, the MATE1 substrate is ganciclovir. In certain embodiments, the MATE1 substrate is guanidine. In certain embodiments, the MATE1 substrate is procainamide.

In certain embodiments, there is provided a method for administering dichlorphenamide, or a pharmaceutically acceptable salt thereof, to a subject in need thereof, wherein the subject is also being administered a therapeutically effective amount of a MATE2-K substrate. The method comprises administering to the subject a therapeutically effective amount of dichlorphenamide, or a pharmaceutically acceptable salt thereof, wherein the therapeutically effective amount of the MATE2-K substrate is not adjusted relative to a subject who is not being administered dichlorphenamide, or a pharmaceutically acceptable salt thereof.

In certain embodiments, there is provided a method for administering dichlorphenamide, or a pharmaceutically acceptable salt thereof, to a subject in need thereof, the method comprising administering to the subject a therapeutically effective amount of dichlorphenamide, or a pharmaceutically acceptable salt thereof, subsequently determining that the subject is to begin treatment with a therapeutically effective amount of a drug chosen from a MATE2-K substrate, and continuing administration of the therapeutically effective amount of dichlorphenamide, or a pharmaceutically acceptable salt thereof, wherein the therapeutically effective amount of the drug is not adjusted relative to a subject who is not being administered dichlorphenamide, or a pharmaceutically acceptable salt thereof.

In certain embodiments, the MATE2-K substrate is chosen from baricitinib, cimetidine, acyclovir, estrone sulfate, ganciclovir, metformin, procainamide, topotecan, and combinations thereof.

In certain embodiments, the MATE2-K substrate is baricitinib. In certain embodiments, the MATE2-K substrate is cimetidine. In certain embodiments, the MATE2-K substrate is acyclovir. In certain embodiments, the MATE2-K substrate is estrone sulfate. In certain embodiments, the MATE2-K substrate is ganciclovir. In certain embodiments, the MATE2-K substrate is metformin. In certain embodiments, the MATE2-K substrate is procainamide. In certain embodiments, the MATE2-K substrate is topotecan.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

In certain embodiments, the method further comprises informing the subject or a medical care worker that administering dichlorphenamide, or a pharmaceutically acceptable salt thereof, to a subject who is also taking a drug chosen from a P-gp substrate, BCRP substrate, OAT2 substrate, OAT4 substrate, OCT1 substrate, MATE1 substrate, MATE2-K substrate, and combinations thereof, results in no increase in drug exposure as compared with administering the drug to a patient who is not being administered dichlorphenamide, or a pharmaceutically acceptable salt thereof.

In certain embodiments, the method further comprises informing the patient or a medical care worker that administering dichlorphenamide, or a pharmaceutically acceptable salt thereof, to a subject who is also taking a drug chosen from a P-gp substrate, BCRP substrate, OAT2 substrate, OAT4 substrate, OCT1 substrate, MATE1 substrate, MATE2-K substrate, and combinations thereof, may result in no increased risk of one or more exposure-related adverse reactions than administering the drug to a subject who is not being administered dichlorphenamide, or a pharmaceutically acceptable salt thereof.

In certain embodiments, the drug is chosen from a P-gp substrate, BCRP substrate, OAT2 substrate, OAT4 substrate, OCT1 substrate, MATE1 substrate, MATE2-K substrate, and combinations thereof. In certain embodiments, the drug is a P-gp substrate. In certain embodiments, the drug is a BCRP substrate. In certain embodiments, the drug is an OAT2 substrate. In certain embodiments, the drug is an OAT4 substrate. In certain embodiments, the drug is a OCT1 substrate. In certain embodiments, the drug is a MATE1 substrate. In certain embodiments, the drug is a MATE2-K substrate.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLE

A study was designed to evaluate dichlorphenamide as an inhibitor of P-gp, BCRP, OAT2, OAT4, OCT1, MATE1 and MATE2-K. Compounds that are substrates or inhibitors of the transporters may be victims or perpetrators in drug-drug interactions. Experiments were carried out as described in the FDA and EMA draft guidance documents for Drug Interaction Studies (FDA 2017, EMA 2013).

Dichlorphenamide was evaluated for its ability to inhibit human ATP-binding cassette transporters (ABC) and solute carrier (SLC) transporters as outlined in the following table. The probe substrates selected for the assays are substrates for the selected transporter and produce a signal sufficient for the detection of inhibition of the transporter.

TABLE 1
Transport, test system, probe substrates and experimental design.

| | Test | | |
|---|---|---|---|
| Transporter | system | Probe substrate | Experimental design |
| P-gp | Caco-2 | Digoxin | Bidirectional transport of |
| | cells | | the probe substrate across |
| BCRP | MDCKII | Prazosin | MDCKII transporter- |
| | cells | | expressing and |
| | | | control MDCKII cells |
| | | | and Caco-2 cells |
| | | | Accumulation of the |
| OAT2 | S₂ cells | [³H]-PGE2 | probe substrate |
| OAT4 | | [³H]-Estrone- | into transporter- |
| | | 3-sulfate | expressing |
| OCT1 | HEK293 | [¹⁴C]-Tetraethyl- | and control cells |
| | cells | ammonium | |
| | | bromide | |
| | | [¹⁴C]-Metformin | |
| MATE1 | | | |
| MATE2-K | | | |

Dichlorphenamide was evaluated as a substrate of human ABC and SLC transporters as outlined in the Table 2. The positive control substrates selected for the assays are substrates for the selected transporter and produce a signal sufficient to detect transporter inhibition.

Caco-2 cells are a polarized cell line derived from a human colon carcinoma that expresses the human ABC transporter P-gp and others. Caco-2 cells were used to evaluate dichlorphenamide and acetazolamide as an inhibitor of P-gp by measuring the effect of dichlorphenamide and acetazolamide on the transport of the P-gp substrate digoxin. Caco-2 cells were purchased from American Type Culture Collection.

Madin Darby Canine Kidney II (MDCKII) cells overexpressing human P-gp and BCRP were used in experiments to evaluate dichlorphenamide and acetazolamide as inhibitors and substrates of P-gp and BCRP. MDCKII P-gp, MDCKII BCRP and control MDCKII cells were purchased from the Netherlands Cancer Institute.

Human embryonic kidney 293 (HEK293) cells expressing transporter transfected with vectors containing human transporter cDNA for OCT1, MATE1 and MATE2-K and control cells (HEK293 cells transfected with only vector) were used in experiments to evaluate dichlorphenamide as inhibitors of OCT1, MATE1 and MATE2-K. HEK293 cells were purchased from American Type Culture Collection and transfected with the transporter gene by Sekisui Medical Co. Ltd. MATE1 and MATE2-K transfected HEK293 cells were purchased from Corning, Inc.

Schneider 2 (S₂) cells are a commonly used Drosophila melanogaster cell lines from a primary culture of late stage (20-24 hours old) embryos from a macrophage-like lineage. S₂ cells are used to express heterologous proteins, to produce proteins on a large scale, and to easily and transiently transfect with several plasmids at once to study protein interactions.

Dichlorphenamide was prepared in dimethyl sulfoxide (DMSO) and spiked into incubation media for a final concentration of 0.1% v/v DMSO. Cells were cultured as described in Table 3. The medium was replaced every 2 to 3 days, and the cells were passaged when confluent.

TABLE 2
Cell cultures.

| | | | Culture | Cell |
|---|---|---|---|---|
| | | | chamber | culture |
| | | | con- | duration |
| Cell | SOP | Cell culture medium | ditions | (days) |
| Caco-2 ^{a} | L5102.04 | EMEM supplemented | 37 ± | 21 |
| | | with FBS (8.9% v/v), | 2° C., | |
| | | non-essential amino | 95 ± 5% | |
| | | acids (0.89% v/v) and | relative | |
| | | penicillin-streptomycin | humidity, | |
| | | (45 U/mL and | and | |
| | | 45 μg/mL, respectively) | 5 ± 1% | |
| MDCKII ^{a} | L5103.03 | DMEM supplemented | CO₂ | 3 to 5 |
| | | with FBS | (in cell | |
| | | (10% v/v) and | culture | |
| | | penicillin-streptomycin | flasks) | |
| | | (45 U/mL and | | |
| | | 45 μg/mL, respectively) | | |
| HEK293 ^{b, c} | L5100.04 | DMEM supplemented | | 1 to 3 |
| | | with FBS (8.9% v/v), | | |
| | | antibiotic/antimycotic | | |
| | | (0.89% v/v) and L- | | |
| | | glutamine (1.79 mM) | | |

^{a}Cells were cultured on a porous membrane in a 24-well transwell plate and allowed to form a confluent monolayer with tight junctions. The monolayer separated the apical and basolateral compartments of the transwell.
^{b}Cells were cultured on a 24-well tissue plate.
^{c}MATE1 and MATE2-K expressing HEK293 cells were thawed and directly plated as specified by the manufacturer.

Non-specific binding of the test articles to the incubation vessels without cells was evaluated by incubating dichlorphenamide in incubation media at low and high concentrations (1 and 1000 μM for dichlorphenamide) in 24-well plates or a 24-well transwell plate at 37±2° C. for either 30 or 120 min. At the end of the incubation period, aliquots of the mixtures were collected, analyzed by LC MS/MS and compared to the dose solutions (100% solution).

Probe substrates and positive control inhibitors were prepared in DMSO at a concentration 1000-fold higher than the incubation concentration and spiked into incubation medium each at 0.1% v/v DMSO. The final concentration of DMSO was 0.2% v/v and was equal in all incubations (e.g., the sum of the DMSO from the probe substrate and dichlorphenamide, positive control inhibitor or the solvent control [DMSO]). The final concentration of DMSO was 0.1% v/v in no solvent control incubations.

Digoxin (12β-hydroxydigitoxin) treat various heart conditions including atrial fibrillation, atrial flutter, and heart failure. Digoxin elimination is mainly by renal excretion and involves P-gp, which leads to significant clinical interactions with P-gp inhibitor drugs. Quinidine, verapamil, and amiodarone increase plasma levels of digoxin by displacing tissue binding sites and depressing renal digoxin clearance.

Prazosin is a sympatholytic medication for treating high blood pressure, anxiety, and posttraumatic stress disorder (PTSD). Prazosin is an α1-blocker that acts as an inverse agonist at alpha-1 adrenergic receptors. Metabolism is primarily hepatic.

Prostaglandin E2 (PGE2, dinoprostone) is a naturally occurring prostaglandin used to induce labor, bleeding after delivery, termination of pregnancy, and in newborn babies to keep the ductus arteriosus open. PGE2 is a potent activator of the Wnt signaling pathway implicated in regulating the developmental specification and regeneration of hematopoietic stem cells through cAMP/PKA activity. PGE2 is rapidly metabolized primarily in the local tissues; any systemic absorption of the medication is cleared mainly in the maternal lungs and, secondarily, at sites such as the liver and kidneys.

Estrone-3-sulfate (estrone sulfate, E1S) is a natural, endogenous steroid and an estrogen ester and conjugate. E1S itself is biologically inactive, with less than 1% of the relative binding affinity of estradiol for the ERα and ERβ, but it can be converted by steroid sulfatase (also called estrogen sulfatase) into estrone, which is an estrogen. Exogenous estrogens are metabolized in the same manner as endogenous estrogens. Circulating estrogens exist in a dynamic equilibrium of metabolic interconversions which occur mainly in the liver.

Metformin (Glucophage™) is the first-line medication for the treatment of type 2 diabetes and polycystic ovary syndrome (PCOS). Metformin decreases high blood sugar, primarily by suppressing liver glucose production (hepatic gluconeogenesis). The H₂-receptor antagonist cimetidine increases the plasma concentration of metformin by reducing clearance of metformin by the kidneys. Both metformin and cimetidine are cleared from the body by tubular secretion, and both, particularly cationic cimetidine, may compete for the same transport mechanism.

Valspodar (PSC833) is an experimental cancer treatment and chemosensitizer. It is a derivative of ciclosporin D (cyclosporine D). Its primary use is as an P-gp inhibitor.

Verapamil treats high blood pressure, angina, supraventricular tachycardia, migraines, and cluster headaches. Verapamil is a P-gp inhibitor. Use of verapamil is generally avoided in people with severe left ventricular dysfunction, hypotension (systolic blood pressure less than 90 mm Hg), cardiogenic shock, and hypersensitivity to verapamil.

Ko143 is a positive control inhibitor BCRP in Michigan Cancer Foundation-7 (MCF7) and BCRP over-expressing MCF7/MX100 cell lines using a BCRP prototypical substrate mitoxantrone. Ko143 displays greater than 200-fold selectivity over P-gp 1 and MRP-1 transporters. It increases intracellular drug accumulation and reverses BCRP-mediated multidrug resistance. It blocked topotecan and albendazole sulfoxide transport in a concentration-dependent manner. Ko143 is a more specific inhibitor of BCRP than other known inhibitors of BCRP such as fumitremorgin C and elacridar (GF120918).

Lopinavir (ABT-378) is an antiretroviral of the protease inhibitor class used against HIV infections as a fixed-dose combination with another protease inhibitor, ritonavir, under the tradenames Kaletra™ (high-income countries) and Aluvia™ (low-income countries). This prevents cleavage of the gag-pol polyprotein and, therefore, improper viral assembly results.

Quinidine is an optical isomer of quinine, extracted from the bark of the Cinchona tree and similar plant species. This alkaloid dampens the excitability of cardiac and skeletal muscles by blocking sodium and potassium currents across cellular membranes, blocks muscarinic and alpha-adrenergic neurotransmission, and inhibits the cytochrome P450 enzyme 2D6, thus increasing blood levels of lidocaine, beta blockers, opioids, and some antidepressants. Quinidine also inhibits the P-gp and can cause peripherally acting drugs such as loperamide to have central nervous system side effects, such as respiratory depression.

Cimetidine (Tagamet™) binds to an H₂-receptor located on the basolateral membrane of the gastric parietal cell, blocking histamine effects. This competitive inhibition reduces gastric acid secretion, gastric volume and acidity. Cimetidine inhibits aminolaevulinic acid synthase (ALA) synthase activity. Due to its non-selective inhibition of cytochrome P450 enzymes, including CYP1A2, CYP2E1, and CYP3A4, and P-glycoprotein, cimetidine has numerous drug interactions. Cimetidine also potently inhibits tubular creatinine secretion.

Pyrimethamine (Daraprim™) is an antiparasitic compound for treating uncomplicated, chloroquine resistant, P. falciparum malaria. Pyrimethamine interferes with the regeneration of tetrahydrofolic acid from dihydrofolate by competitively inhibiting dihydrofolate reductase. Other antifolate agents such as methotrexate and trimethoprim may potentiate the antifolate actions of pyrimethamine, leading to potential folate deficiency, anemia, and other blood dyscrasias.

Caco-2 cells were cultured on 24-well transwell plates for 21 days and or MDCKII cells were cultured for 3 to 5 days, before the experiment. After cell culture, culture medium was removed, and incubation medium was added to the cells. About 10 min after incubation medium was added, transepithelial/transendothelial electric resistance (TEER) values were recorded to determine cytotoxicity and cells were preincubated at 37±2° C. for 30 to 60 min. After preincubation, incubation medium with probe substrate containing the solvent control, control inhibitor, dichlorphenamide was added to the donor chamber and incubation medium containing the solvent control, control inhibitors, dichlorphenamide was added to the receiver chamber for total incubation volumes of 200 and 980 μL for the apical and basolateral chambers, respectively. Samples (100 μL) were collected from the receiver compartment at 120 min. In wells in which the recovery was calculated, samples (20 μL) were taken from the donor chambers at the start of the incubation (time zero) and after the final time point (120 min). If the donor chamber was sampled at time zero, the volume added to the donor chamber at time zero was 20 μL higher (220 or 1000 μL). Samples containing the probe substrate were mixed with internal standard and analyzed by LC MS/MS.

The ability of dichlorphenamide to inhibit the accumulation of probe substrates into transporter-expressing and control cells was measured to evaluate dichlorphenamide and acetazolamide as inhibitors of SLC transporters. Inhibition of transporters was determined by incubating the cells with a probe substrate and dichlorphenamide or acetazolamide and measuring the amount of probe substrate accumulating in the cells.

Radiolabeled substrates, except for [¹⁴C]-metformin, were dried under a stream of nitrogen then reconstituted in non-labeled substrate or solvent. [¹⁴C]-Metformin (1 mM) was provided as a solid and was prepared in Hank's balanced salt solution (HBSS). Probe substrates and positive control inhibitors were prepared in DMSO at a concentration 1000-fold higher than the incubation concentration and spiked into incubation medium each at 0.1% v/v DMSO. The final concentration of DMSO was 0.2% v/v and was equal in all incubations. That is, the sum of the DMSO from the probe substrate and dichlorphenamide, positive control inhibitor or the solvent control (DMSO) were equal. The final concentration of DMSO was 0.1% v/v in no solvent control incubations.

Cells were plated onto standard 24-well tissue culture plates in cell culture medium 1 to 3 days before the experiment. MATE1 and MATE2-K and control cells were incubated with butyric acid (10 mM) for 24 hours before the experiment to inhibit suppression of the transporter. Incubations of HEK293 cells were performed in HBSS buffer containing sodium bicarbonate (4 mM) and HEPES (9 mM), pH 7.4 (OAT and OCT) or pH 8.5 (MATE).

After incubation, incubation medium was removed, and cells were rinsed once with 1 mL of ice-cold phosphate-buffered saline (PBS) containing 0.2% w/v bovine specific antigen (BSA) and twice with ice-cold PBS. The PBS was removed, and 0.5 mL of sodium hydroxide (0.1 M) was added and pipetted up and down to dissolve and suspend the cells. An aliquot of the medium was added to a 96 well plate, diluted with scintillation fluid and analyzed on a MicroBeta2 scintillation counter. The amount of protein in each incubation was determined by bicinchoninic acid (BCA) analysis.

Tables 3-7 show the that dichlorphenamide was not an inhibitor of P-gp, BCRP, OAT2, OAT4, OCT1, MATE1 and MATE2-K (IC₅₀>1000 μM). Where applicable, n is the number of replicates, NA is Not applicable, and SD refers to the standard deviation. Unless otherwise noted, values are triplicate determinations rounded to three significant figures with standard deviations rounded to the same degree of accuracy. Percentages are rounded to one decimal place except percentages>100, which are rounded to the nearest whole number.

TABLE 3
Dichlorphenamide: P-gp inhibition across Caco-2 cells using
digoxin 10 μM for the substrate.

| | | Pₐₚₚ (×10⁻⁶ cm/sec) | | | | |
|---|---|---|---|---|---|---|
| | [Inhibitor] | (Average ± SD) | | | | IC₅₀ |
| Inhibitor | (μM) | A:B | B:A | Efflux ratio | % control | parameters |
| No solvent | 0 | 1.34 ± 0.44 | 28.9 ± 2.3 | 21.6 | NA | NA |
| control | | | | | | |
| Solvent | 0 | 0.813 ± 0.307 | 23.2 ± 4.9 | 28.5 | 100 | IC₅₀: |
| control | | | | | | >1000 μM |
| Dichlor- | 3 | 1.34 ± 0.55 | 28.5 ± 1.1 | 21.4 | 74.0 | |
| phenamide | 10 | 0.837 ± 0.099 | 23.5 ± 2.4 | 28.0 | 98.2 | |
| | 30 | 0.957 ± 0.235 | 24.6 ± 4.3 | 25.7 | 89.8 | |
| | 100 | 1.30 ± 0.23 | 29.6 ± 1.5 | 22.8 | 79.2 | |
| | 300 | 0.337 (n = 2) | 25.9 ± 1.9 | 76.8 | 275 | |
| | 1000 | 0.739 ± 0.195 | 26.5 ± 4.6 | 35.8 | 127 | |
| Valspodar | 1 | 9.12 ± 1.34 | 10.0 ± 2.5 | 1.10 | 0.4 | NA |
| Verapamil | 60 | 7.09 ± 4.10 | 8.66 ± 1.29 | 1.22 | 0.8 | |

| | | Recovery (%) | |
|---|---|---|---|
| | | A:B | B:A |
| No solvent | 0 | 90.6 | 88.9 |
| control | | | |
| Dichlor- | 0 | 88.9 | 89.0 |
| phenamide | 1000 | 88.9 | 90.6 |

A:B = apical to basal ratio; B:A = basal to apical ratio

TABLE 4
Dichlorphenamide: BCRP inhibition across MDCKII cells using a
Prazosin (1 μM) for the substrate.

| | | Control cells | | | MDCKII-BCRP cells | | |
|---|---|---|---|---|---|---|---|
| | | Pₐₚₚ (×10⁻⁶ cm/sec) | | | Pₐₚₚ (×10⁻⁶ cm/sec) | | |
| | | (Average ± SD) | | | (Average ± SD) | | |
| | [Inhibitor | Apical to | Basal to | Efflux | Apical to | Basal to | Efflux |
| Inhibitor | 1 (μM) | basal | apical | ratio | basal | apical | ratio |
| No solvent | 0 | 28.8 ± 8.6 | 28.9 ± 2.0 | 1.00 | 10.0 ± 1.9 | 74.7 ± 5.9 | 7.44 |
| control | | | | | | | |
| Solvent | 0 | 18.3 ± 12.0 | 20.6 ± 2.6 | 1.12 | 10.5 ± 0.8 | 69.7 ± 4.6 | 6.66 |
| control | | | | | | | |
| Dichlor- | 3 | 25.8 ± 4.9 | 24.9 ± 3.5 | 0.963 | 9.60 ± 0.51 | 65.6 ± 4.2 | 6.83 |
| phen- | 10 | 18.7 ± 12.2 | 18.7 ± 3.3 | 1.00 | 10.7 ± 2.1 | 58.9 ± 9.5 | 5.51 |
| amide | 30 | 16.7 ± 10.8 | 17.9 ± 3.8 | 1.07 | 10.8 ± 1.0 | 59.5 ± 4.0 | 5.49 |
| | 100 | 19.6 ± 8.3 | 19.4 ± 2.3 | 0.990 | 12.2 ± 0.9 | 66.2 ± 7.9 | 5.44 |
| | 300 | 22.0 ± 4.7 | 22.8 ± 4.2 | 1.04 | 12.7 ± 0.7 | 73.0 ± 5.7 | 5.73 |
| | 1000 | 15.4 ± 7.6 | 17.5 ± 2.3 | 1.14 | 13.2 ± 1.5 | 59.5 ± 3.9 | 4.51 |
| Ko143 | 1 | 24.6 ± 2.1 | 25.0 ± 1.6 | 1.02 | 35.8 | 38.3 ± 0.6 | 1.07 |
| | | | | | (n = 2) | | |
| Lopinavir | 30 | 13.3 ± 10.0 | 20.9 ± 4.7 | 1.57 | 32.3 | 37.6 ± 1.6 | 1.17 |
| | | | | | (n = 2) | | |

| Corrected efflux ratio | | | % control | IC₅₀ parameters |
|---|---|---|---|---|
| No solvent | 0 | 7.41 | NA | NA |
| control | | | | |
| Solvent | 0 | 5.93 | 100 | IC₅₀: >1000 μM |
| control | | | | |
| Dichlor- | 3 | 7.10 | 124 | |
| phen- | 10 | 5.51 | 91.4 | |
| amide | 30 | 5.11 | 83.3 | |
| | 100 | 5.50 | 91.2 | |
| | 300 | 5.53 | 91.9 | |
| | 1000 | 3.96 | 60.0 | |
| Ko143 | 1 | 1.05 | 1.0 | NA |
| Lopinavir | 30 | 0.742 | 0 | |

| | | Control cells | | MDCKII-BCRP cells | |
|---|---|---|---|---|---|
| | | Recovery (%) | | Recovery (%) | |
| | | A:B | B:A | A:B | B:A |
| No solvent | 0 | 82.0 | 83.1 | 82.8 | 87.5 |
| control | | | | | |
| Dichlor- | 0 | 75.5 | 90.2 | 81.9 | 86.2 |
| phen- | 1000 | 84.7 | 77.0 | 93.0 | 83.7 |
| amide | | | | | |

A:B = apical to basal ratio; B:A = basal to apical ratio

TABLE 5
Dichlorphenamide: OCT1 inhibition in HEK293 cells using
[¹⁴C]-Tetraethylammonium bromide (5 μM) for the probe substrate

| | | Uptake (pmol/mg | | Background | | |
|---|---|---|---|---|---|---|
| | | protein) | | corrected | | |
| | [Inhibitor] | (Average ± SD) | | uptake rate | | IC₅₀ |
| Inhibitor | (μm) | Control | OCT1 | (pmol/mg/min) | % control | parameters |
| No solvent | 0 | 3.96 ± 0.28 | 115 ± 3 | 7.38 | NA | NA |
| control | | | | | | |
| Solvent | 0 | 4.12 ± 0.42 | 122 ± 11 | 7.87 | 100 | IC₅₀: |
| control | | | | | | >1000 μM |
| Dichlor- | 1 | 4.28 ± 1.48 | 121 ± 11 | 7.76 | 98.6 | |
| phenamide | 3 | 4.44 ± 1.97 | 125 ± 5 | 8.07 | 102 | |
| | 10 | 4.28 ± 0.92 | 111 ± 4 | 7.13 | 90.6 | |
| | 30 | 3.88 ± 0.48 | 122 ± 14 | 7.85 | 99.7 | |
| | 100 | 4.12 ± 0.73 | 128 ± 6 | 8.26 | 105 | |
| | 300 | 3.88 ± 1.11 | 130 ± 10 | 8.40 | 107 | |
| | 1000 | 2.99 ± 1.46 | 77.9 ± 3.0 | 5.00 | 63.5 | |
| Quinidine | 100 | 2.26 ± 0.78 | 26.8 ± 2.5 | 1.64 | 20.8 | NA |
| Verapamil | 10 | 3.07 ± 0.98 | 46.5 ± 2.0 | 2.90 | 36.8 | |

TABLE 6
Dichlorphenamide: MATE1 inhibition
in HEK293 cells using for the probe substrate

| | | Uptake (pmol/mg | | Background | | |
|---|---|---|---|---|---|---|
| | | protein) | | corrected | | |
| | [Inhibitor] | (Average ± SD) | | uptake rate | | IC₅₀ |
| Inhibitor | (μm) | Control | MATE1 | (pmol/mg/min) | % control | parameters |
| No solvent | 0 | 7.39 ± 1.03 | 414 ± 20 | 81.3 | NA | NA |
| control | | | | | | |
| Solvent | 0 | 6.88 ± 0.42 | 421 ± 15 | 82.8 | 100 | IC₅₀: |
| control | | | | | | >1000 μM |
| Dichlor- | 1 | 6.66 ± 1.31 | 436 ± 23 | 85.8 | 104 | |
| phenamide | 3 | 6.75 ± 1.65 | 401 ± 7 | 78.8 | 95.2 | |
| | 10 | 7.43 ± 1.34 | 406 ± 12 | 79.8 | 96.4 | |
| | 30 | 6.79 ± 0.89 | 432 ± 10 | 85.0 | 103 | |
| | 100 | 7.52 ± 0.96 | 421 ± 17 | 82.6 | 99.8 | |
| | 300 | 5.56 ± 0.16 | 398 ± 17 | 78.4 | 94.7 | |
| | 1000 | 5.52 ± 0.21 | 379 ± 13 | 74.7 | 90.3 | |
| Cimetidine | 10 | 5.20 ± 0.63 | 85.2 ± 7.8 | 16.0 | 19.3 | NA |
| Pyrimeth- | 0.1 | 4.24 ± 0.72 | 62.8 ± 4.0 | 11.7 | 14.2 | |
| amine | | | | | | |

TABLE 7
Dichlorphenamide: MATE2-K inhibition in HEK293 cells
using [¹⁴C]-Metformin (10 μM) for the probe substrate.

| | | Uptake (pmol/mg | | | | |
|---|---|---|---|---|---|---|
| | | protein) | | Background | | |
| | | (Average ± SD) | | corrected | | |
| | [Inhibitor] | | MATE2- | uptake rate | % | IC₅₀ |
| Inhibitor | (μM) | Control | K | (pmol/mg/min) | control | parameters |
| No solvent | 0 | 9.56 ± 1.85 | 134 ± 12 | 24.9 | NA | NA |
| control | | | | | | |
| Solvent | 0 | 7.34 ± 1.46 | 146 ± 13 | 27.6 | 100 | IC₅₀: |
| control | | | | | | >1000 μM |
| Dichlorphen | 1 | 9.21 ± 0.93 | 149 ± 29 | 28.0 | 101 | |
| amide | 3 | 9.44 ± 2.27 | 159 ± 19 | 29.9 | 108 | |
| | 10 | 8.28 ± 1.46 | 113 ± 8 | 21.0 | 76.1 | |
| | 30 | 8.51 ± 1.05 | 124 ± 36 | 23.1 | 83.7 | |
| | 100 | 9.21 ± 1.52 | 137 ± 15 | 25.5 | 92.4 | |
| | 300 | 8.86 ± 0.93 | 166 ± 16 | 31.4 | 114 | |
| | 1000 | 5.13 ± 0.40 | 106 ± 29 | 20.2 | 73.2 | |
| Cimetidine | 300 | 7.34 ± 0.20 | 32.3 ± 11.7 | 4.99 | 18.1 | NA |
| Pyrimeth- | 0.3 | 5.59 ± 0.88 | 75.6 ± 16.7 | 14.0 | 50.6 | |
| amine | | | | | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for administering dichlorphenamide, or a pharmaceutically acceptable salt thereof, to a subject in need thereof, wherein the subject is also being administered a therapeutically effective amount of a OAT2 substrate, the method comprising:
administering to the subject a therapeutically effective amount of dichlorphenamide, or a pharmaceutically acceptable salt thereof,
wherein the dichlorphenamide, or a pharmaceutically acceptable salt thereof, is administered to the subject to treat a disease chosen from primary hyperkalemic periodic paralysis, primary hypokalemic periodic paralysis, and related variants, and
wherein the therapeutically effective amount of the OAT2 substrate is not adjusted relative to a subject who is not being administered dichlorphenamide, or a pharmaceutically acceptable salt thereof.

2. A method for administering dichlorphenamide, or a pharmaceutically acceptable salt thereof, to a subject in need thereof, the method comprising:
administering to the subject a therapeutically effective amount of dichlorphenamide, or a pharmaceutically acceptable salt thereof,
subsequently determining that the subject is to begin treatment with a therapeutically effective amount of a OAT2 substrate, and
continuing administration of the therapeutically effective amount of dichlorphenamide, or a pharmaceutically acceptable salt thereof, and beginning administration of the therapeutically effect amount of the OAT2 substrate,
wherein the dichlorphenamide, or a pharmaceutically acceptable salt thereof, is administered to the subject to treat a disease chosen from primary hyperkalemic periodic paralysis, primary hypokalemic periodic paralysis, and related variants, and
wherein the therapeutically effective amount of the OAT2 substrate is not adjusted relative to a subject who is not being administered dichlorphenamide, or a pharmaceutically acceptable salt thereof.

3. The method claim 1, further comprising informing the subject or a medical care worker that administering dichlorphenamide, or a pharmaceutically acceptable salt thereof, to a subject who is also taking a OAT2 substrate, results in no increase in drug exposure as compared with administering the OAT2 substrate to a subject who is not being administered dichlorphenamide, or a pharmaceutically acceptable salt thereof.

4. The method of claim 1, further comprising informing the subject or a medical care worker that administering dichlorphenamide, or a pharmaceutically acceptable salt thereof to a subject who is also taking a OAT2 substrate, may result in no increased risk of one or more exposure-related adverse reactions than administering the OAT2 substrate to a subject who is not being administered dichlorphenamide, or a pharmaceutically acceptable salt thereof.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. The method of claim 1, wherein the therapeutically effective amount of the dichlorphenamide, or a pharmaceutically acceptable salt thereof, is between 25 mg and 200 mg per day.

21. The method of claim 1, wherein the therapeutically effective amount of the dichlorphenamide, or a pharmaceutically acceptable salt thereof, is 50 mg twice daily.

22. The method of claim 1, wherein the dichlorphenamide, or a pharmaceutically acceptable salt thereof, is administered via a titration scheme that comprises the up-titration of the dichlorphenamide, or a pharmaceutically acceptable salt thereof, at weekly intervals until a modified dose is administered.
